(19) United States
(12) Patent Application Publication
(10) Pub. No.: US 2025/0353871 A1
(43) Pub. Date: Nov. 20, 2025

(54) PRODUCTION OF GLYCOSYL FLUORIDES

(71) Applicant: Carbocode S.A., Coimbra (PT)

(72) Inventors: Halina ZHYLITSKAYA, Coimbra (PT); Katharina KETTELHOIT, Coimbra (PT); Gyula DEKANY, Coimbra (PT); Györgyi OSZTROVSZKY, Coimbra (PT); Ferenc HORVATH, Coimbra (PT)

(21) Appl. No.: 18/866,488

(22) PCT Filed: May 16, 2023
(86) PCT No.: PCT/EP2023/063189

§ 371 Date: Nov. 15, 2024

(30) Foreign Application Priority Data
May 17, 2022 (PT) 117981
Sep. 16, 2022 (PT) 118199

(51) Int. Cl. C07H 15/04; C07H 5/02; C07H 15/18

(52) U.S. Cl. CPC C07H 15/04; C07H 5/02; C07H 15/18

(57) ABSTRACT

The present invention relates to a novel and efficient method for the production of glycosyl fluorides by the fluorination of a protected saccharide with a fluorinating agent, such as poly(hydrogen fluoride), triethylamine trihydrofluoride, and DMPU-HF, wherein the fluorination is performed in the presence of a Lewis acid.

### FIELD OF THE INVENTION

The present invention relates to a method for producing glycosyl fluorides.

### BACKGROUND

Glycosyl fluorides such as 1-deoxy-1-fluoro glycosides are carbohydrate derivatives which can be described as α-halo ethers. They can be obtained in both anomeric forms, but the α-anomer is the more stable.

Glycosyl fluorides are important building blocks for the synthesis of complex oligosaccharides, and display a remarkable stability. In fact, they are the only glycosyl halide which can be dissolved in water. Furthermore, most glycosyl fluorides are crystalline compounds and can be stored for a long time without decomposition.

Owing to their stability this class of compounds has found many applications in chemistry, biochemistry, and biotechnology.

Particularly, glycosyl fluorides have been used for the synthesis of glycosphingolipids, wherein a glycosyl fluoride donor is coupled to D-erythro-sphingosine in the presence of an endoglycoceramidase glycosynthase (EGCase) (M. D. Vaughan et al. J. Am. Chem. Soc., 2006, 128, 6300-6301). Furthermore, glycosyl fluorides can be internalized by engineered cells and converted into complex glycosyl fluorides with applications in pharma and biology (WO 2021/170620 A1).

Accordingly, synthetic access to glycosyl fluoride may enable the production of biologically relevant compounds, such as for example glycosphingolipids.

Chemical synthesis is required for the installation of a fluoride onto the anomeric carbon of a saccharide. Several methods are available for the anomeric fluorination of saccharides, wherein a protected saccharide is reacted with a fluorinating agent such as for example hydrogen fluoride, pyridinium polyhydrogen fluoride, DAST, Xtalfluor, deoxofluor etc. (Uhrig et al., Org. Biomol. Chem., 2019, 17, 5173-5189). Drawbacks connected to these methods, comprise the use of large amounts of fluorinating agents such as hydrogen fluoride (DE 4021001 A1), or pyridinium polyhydrogen fluoride (J. Junneman et al., Carbohydr. Res. 1993, 249, 91-94), or the use of unstable, and/or expensive fluorinating agents, such as DAST, Xtalfluor, or deoxofluor, rendering the synthesis of glycosyl fluorides difficult to scale up.

Therefore, there is a demand for the development of novel methodologies characterized by high technological feasibility and low costs, which enable the efficient and large-scale production glycosyl fluorides with application in the synthesis of biologically relevant compounds.

### SUMMARY OF THE INVENTION

In a first aspect the present invention relates to a method for producing a glycosyl fluoride by the fluorination of a protected saccharide, wherein each hydroxyl group of said protected saccharide is derivatized with a protecting group, and wherein the anomeric hydroxyl group of said protected saccharide is derivatized with an acyl protecting group, the method comprising the steps of:
reacting the protected saccharide with a fluorinating agent, wherein the fluorinating agent is selected from the group consisting of pyridinium poly(hydrogen fluoride), triethylamine trihydrofluoride, and DMPU-HF, thereby obtaining a protected glycosyl fluoride,
deprotecting the protected glycosyl fluoride, thereby producing the glycosyl fluoride,
   and wherein the step of reacting the protected saccharide with the fluorinating agent is performed in the presence of a Lewis acid.

### DETAILED DESCRIPTION OF INVENTION

The present inventors have found that surprisingly, glycosyl fluorides can be produced under conditions which are mild and do not require the use of a large excess (e.g. about 40 molar equivalents) of a fluorinating agent such as pyridinium poly(hydrogen fluoride).

Particularly, the present inventors have found that a stoichiometric amount or a slight excess (e.g. from about 1 to about 10 molar equivalents) of the fluorinating agent is sufficient when the fluorination reaction is performed in the presence of a Lewis acid. Therefore, the method described herein is particularly suitable for the industrial-scale production of glycosyl fluorides from protected saccharides, wherein each hydroxyl group of said protected saccharide is derivatized with a protecting group, and wherein the anomeric hydroxyl group of said protected saccharide is derivatized with an acyl protecting group,

the method comprising the following steps:
reacting the protected saccharide with a fluorinating agent, wherein the fluorinating agent is selected from the group consisting of pyridinium poly(hydrogen fluoride), triethylamine trihydrofluoride, and DMPU-HF, thereby obtaining a protected glycosyl fluoride,
deprotecting the protected glycosyl fluoride, thereby producing the glycosyl fluoride.

Non-limiting embodiments of different aspects of the invention are described below and illustrated by non-limiting examples.

The terms, definitions and embodiments described throughout the specification of the invention relate to all aspects and embodiments of the invention.

The term “a” grammatically is a singular, but it may as well mean the plural of e.g., the intended compound. For example, a skilled person would understand that in the expression “a fluorinating agent”, the provision of not only one single fluorinating agent, but of a variety of fluorinating agents of the same type is meant.

As used herein, the term “acyl” refers to a group derived by the removal of one or more hydroxyl group from an oxoacid, preferably from a carboxylic acid. The acyl group according to the present invention is typically a saturated or unsaturated C₂₋₆ acyl, which may be substitute or unsubstituted.

In the context of the present invention, the terms “about”, “around”, or “approximate” are applied interchangeably to a particular value (e.g. “a temperature of about 5° C.”, “a temperature of around 5° C.”, or “a temperature of approximate 5° C.”), or to a range (e.g. “an amount from about 1 to about 10 “an amount from around 1 to around 10”, or “an amount from approximate 1 to approximate 10”), to indicate a deviation from 0.1% to 10% of that particular value.

As used herein, the term “fluorination” refers to a chemical reaction wherein a fluorine is introduced into an organic molecule. Typically, in the context of the present invention, a fluorination reaction refers to a chemical reaction which results in the replacement of the carbon-oxygen bond at the anomeric position of a protected saccharide by a carbon-fluorine bond.

The term “O-acylation”, as used herein, refers to an esterification reaction wherein the hydroxyl groups of a saccharide react with an organic acid anhydride, or an acyl chloride to form an acyl ester or an acylate.

In connection with the term O-acylation the term “stereoselective” refers to an O-acylation reaction which results in the preferential formation of one stereoisomer among a mixture of stereoisomers. The preferred stereoisomer may be the only product of the reaction or may be formed as component of an unequal mixture of stereoisomers. Typically, in the contest of the present invention an O-acylation reaction is considered stereoselective when the preferred stereoisomer constitutes at least about 55% of the mixture of stereoisomers, preferably about 60%, 65%, 70%, 75%, 80%, 85%, 90%, 91%, 92%, 93%, 94%, 95%, 96%, 97%, 98%, 99% or higher.

As used herein, the expression “the reaction is conducted under reflux” refers to a reaction wherein the mixture of reactant and solvent is heated at about the temperature at which the solvent boils, and the vapours generated from the reaction mixture are condensed back into the reaction vessel.

As used herein, the term “aprotic solvent” refers to any solvent which lacks a labile (acidic) hydrogen atom. The aprotic solvent may be a polar aprotic solvent, or a non-polar aprotic solvent. Polar aprotic solvents are characterized by a net positive dipole moment, and a relatively high dielectric constant. Examples of polar aprotic solvents include, but are not limited to, hydrofurans (e.g. tetrahydrofuran, etc.), hydropyrans, organic esters (e.g. ethylacetate, propylacetate, butyl acetate, etc.), ketones (e.g. acetone, methyl-ethyl ketone, methyl-isobutyl ketone, etc.), dichloromethane, dimethylformamide, acetonitrile, propionitrile, dimethylsulfoxide, propylene carbonate, N-methyl-2-pyrrolidone, and the like. Non-polar aprotic solvents are characterized by a low dielectric constant and are not miscible with water. Examples of non-polar solvents include, but are not limited to alkane (e.g. hexane, heptane, cyclohexane, etc.), aromatic hydrocarbons (e.g. toluene, xylene, mesitylene etc.) ethers (e.g. dioxane, methyl-tertbutyl ether, diisopropyl ether, etc.), and the like.

The term “1-β-glycosyl ester”, as used herein, refers to an O-acylated derivative of a saccharide, wherein at least the anomeric hydroxyl group carries an acyl group, and wherein the anomeric configuration is β.

As used herein, the term “fluorinating agent” refers to a nucleophilic fluorinating agent which can convert a carbon-oxygen bond to a carbon-fluorine bond.

The term “Lewis acid” denote, in the context of the present invention, substances that can accept a pair of nonbonding electrons.

The term “protecting group” refers to a group which has been introduced onto a functional group in a compound, and which modifies the chemical reactivity of said functional group. Typically, the protecting group modifies the chemical reactivity of the functional group in such a way that it renders said functional group chemically inert to the reaction conditions used when a subsequent chemical transformation is performed on said compound.

The person skilled in the art would understand that a protecting group is introduced onto a functional group of a compound through the reaction between the (unprotected) functional group and a protecting group precursor, therefore generating a “protected” derivative of said compound, such as a protected saccharide.

The term, “glycosyl moiety of a ganglioside” as used herein is defined to encompass glycosyl moieties, wherein the anomeric carbon at the reducing end of the oligosaccharide portion of the ganglioside is engaged in a glycosidic bond with another chemical entity, such as a fluoride. The glycosidic bond may be an alpha or a beta glycosidic bond, preferably an alpha glycosidic bond.

The term “protected glycosyl moiety” as used herein, refers to protected derivative of a glycosyl moiety wherein all hydroxyl groups of said glycosyl moiety are derivatized with a protecting group. The hydroxyl groups of the protected glycosyl moiety may all be derivatized with the same protecting group or may be each independently derivatized with a different protecting group. Suitable protecting groups for use in the context of the present invention are protecting groups that are inert under the conditions of the fluorination reaction. Examples of suitable protecting groups include but are not limited to acyl, benzoyl, benzyl, alkylsilyloxy, alkyloxy et cetera.

The term “saccharide”, as used herein refers to a monosaccharide, a disaccharide, or an oligosaccharide (more than one monosaccharide units). A saccharide having more than one monosaccharide unit may represent a linear or a branched structure.

The monosaccharide unit can be any C₅₋₉ sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

In some embodiments, the saccharide is a saccharide of formula (2):

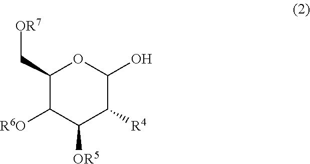

wherein,
R⁴ is selected —NHAc, or —OR⁸ wherein R⁸ is selected from hydrogen or a glycosyl moiety,
R⁵, R⁶, and R⁷, are independently selected from hydrogen or a glycosyl moiety.

In some embodiments, the saccharide of formula (2) is a saccharide of formula (3):

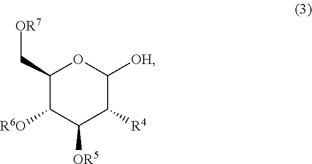

wherein
R⁴, R⁵, R⁶, and R⁷ are as defined as for the saccharide of formula (2).

In some embodiments, the saccharide of formula (2) is a saccharide of formula (4):

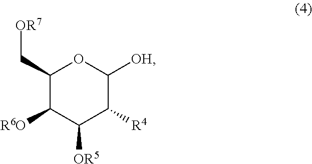

wherein
R⁴, R⁶, R⁶, and R⁷ are as defined as for the saccharide of formula (2).

In some embodiments, for the saccharide of formula (2), (3), or (4) R⁴ is —OH, R⁵ is hydrogen, R⁶ is selected from hydrogen or a glycosyl moiety, and R⁷ is hydrogen.

In some embodiments, the saccharide of formula (2) is a saccharide of formula (3), wherein R⁴ is —OH, R⁵ and R⁶ are hydrogens, and R⁷ is a glycosyl moiety.

In some embodiments for the saccharide of formula (2), or (3) R⁶ is hydrogen.

In some embodiments, for the saccharide of formula (2), or (3) R⁶ is a glycosyl moiety selected from the group consisting of Galβ1-, Galβ1-3GlcNAcβ1-3Galβ1-, Galβ1-4GlcNAcβ1-3Galβ1-.

In some embodiments, the saccharide of formula (2), or (3) is glucose.

In some embodiments, the saccharide of formula (2), or (3) is lactose.

In some embodiments, the saccharide of formula (2), or (3) is lacto-N-tetraose.

In some embodiments, the saccharide of formula (2) or (3) is lacto-N-neotetraose.

In some embodiments, the saccharide of formula (4) is galactose.

In some embodiments, the saccharide of formula (2) is melibiose.

Saccharides such as galactose, glucose, lactose, lactose-N-tetraose, lacto-N-neotetraose, and melibiose are commercially available and can be purchased from established manufacturer.

The term “protected saccharide”, as used herein refers to a protected derivative of a monosaccharide, a disaccharide, or an oligosaccharide (more than one monosaccharide units) wherein all hydroxyl groups of said saccharide are derivatized with a protecting group. A protected saccharide having more than one monosaccharide unit may represent a linear or a branched structure.

The monosaccharide unit can be any C₅₋₉ sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

The hydroxyl groups of the saccharide may be all derivatized with the same protecting group or may be each independently derivatized with different protecting groups. Suitable protecting groups for use in the context of the present invention are protecting groups that are inert under the conditions of the fluorination reaction. Examples of suitable protecting groups include but are not limited to acyl, benzoyl, benzyl, alkylsilyloxy, alkyloxy etc.

Protected saccharides suitable for use in the context of the present invention are those wherein each hydroxyl group is derivatized with a protecting group, and wherein the anomeric hydroxyl group at the reducing end of said protected saccharide is derivatized with an acyl protecting group.

In some embodiments, all the hydroxyl groups of the saccharide are derivatized with an acyl protecting group, wherein the acyl protecting group is selected from the group consisting of a C₂₋₆ acyl, or a benzoyl protecting group.

In some embodiments, all hydroxyl groups of the saccharide are derivatized with an acyl protecting group, wherein the acyl protecting group is preferably a C₂₋₆ acyl. Accordingly in some embodiments the protected saccharide is a per-O-acylated saccharide.

In some embodiments, all hydroxyl groups of the saccharide are derivatized with an acetyl protecting group. Accordingly in some embodiments the protected saccharide is a per-O-acetylated saccharide.

The protected saccharide may be an alpha (α) or a beta (β) saccharide, preferably is a beta (β) saccharide.

In some embodiments the protected saccharide is glucose pentaacetate, galactose pentaacetate, or lactose octaacetate, wherein the protected saccharide may be an alpha (α) or a beta (β) saccharide, preferably is a beta (β) saccharide.

In some embodiments, the protected saccharide is β-D-lactose octaacetate.

In some embodiments, the protected saccharide is β-D-glucose pentaacetate.

In some embodiments, the protected saccharide is β-D-galactose pentaacetate.

In some embodiments, the protected saccharide is β-D-melibiose octaacetate.

In some embodiments, the protected saccharide is a protected derivative of the oligosaccharide portion of a ganglioside preferably selected from GM1a, GM1b, GD1a, GD1b, GD3, GT1b, GT3, GQ1b, GM3, GM4, wherein all hydroxyl groups of the oligosaccharide portion of the ganglioside are derivatized with an acyl protecting group, and wherein for those oligosaccharide portions of gangliosides carrying a sialic acid unit, such as a N-acetylneuraminic acid unit, the carboxylic acid function of the sialic acid unit is in the form of an alkyl ester.

In some embodiments, all hydroxyl groups of the oligosaccharide portion of the ganglioside are derivatized with an acetyl protecting group.

In some embodiments, the protected saccharide is a human milk oligosaccharide preferably selected from LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT, wherein all hydroxyl groups of the human milk oligosaccharide are derivatized with an acyl protecting group, and wherein for those oligosaccharides carrying a sialic acid unit, such as N-acetylneuraminic acid unit, the carboxylic function of the sialic acid unit is in the form of an alkyl ester.

In some embodiments all hydroxyl groups of the human milk oligosaccharide are derivatized with an acetyl protecting group.

In some embodiments, the protected saccharide is β-D-lacto-N-neotetraosyl tetradecaacetate. In some embodiments, the protected saccharide is β-D-lacto-N-tetraosyl tetradecaacetate.

As used herein, the term “glycosyl fluorides” refers to a 1-deoxy-1-fluoro glycoside wherein a fluoride is covalently attached to the anomeric carbon of the reducing end of a glycosyl moiety.

The fluoride may be bound to the anomeric carbon of the glycosyl moiety by either an alpha (α) or a beta (β) glycosidic linkage. An alpha (α) glycosidic linkage is preferred.

The glycosyl moiety of the glycosyl fluoride may derive from a monosaccharide or from an oligosaccharide (more than one monosaccharide units). A glycosyl moiety having more than one monosaccharide unit may represent a linear or a branched structure.

The monosaccharide unit can be any C₅₋₉ sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

The glycosyl moieties according to the present invention may be illustrated in the following style: Galβ1-4Glc1-, wherein the dash (-) represents the point of attachment of the glycosyl moiety and wherein the glycosyl moiety may be linked via an alpha or a beta glycosidic bond, preferably an alpha glycosidic bond.

In some embodiments, the glycosyl moiety of the glycosyl fluoride is that of glucose, galactose, and lactose, wherein the glycosyl moiety may be linked via an alpha (α) or a beta (β) glycosidic bond, preferably an alpha (α) glycosidic bond. In the context of the present invention glycosyl fluorides wherein the glycosyl moiety is that of glucose, galactose, lactose, and melibiose may be represented by the following formulas: Glc1-F, Gal1-F, Galβ1-4Glc1-F, and Galα1-6Glc1-F respectively.

In some preferred embodiments, the glycosyl fluoride is α-D-lactopyranosyl fluoride.

In some embodiments, the glycosyl fluoride is α-D-glucopyranosyl fluoride.

In some embodiments, the glycosyl fluoride is α-D-galactopyranosyl fluoride.

In some embodiments, the glycosyl fluoride is α-D-melibiosyl fluoride.

In some embodiments, the glycosyl moiety of the glycosyl fluoride is the glycosyl moiety of a ganglioside selected from GM1a, GM1b, GD1a, GD1b, GD3, GT1b, GT3, GQ1b, GM3, GM4.

In the context of the present invention glycosyl fluorides wherein the glycosyl moiety is that of GM1a, GM1b, GD1a, GD1b, GD3, GT1b, GT3, GQ1b, and GM4 may be represented by the following formulas:

Galβ1-3GalNAcβ1-4(Neu5Acα2-3)Galβ1-4Glc1-F,
Neu5Acα2-3Galβ1-3GalNAcβ1-4Galβ1-4Glc1-F,
Neu5Acα2-3Galβ1-3GalNAcβ1-4(Neu5Acα2-3)Galβ1-4Glc1-F,
Galβ1-3GalNAcβ1-4(Neu5Acα2-8Neu5Acα2-3)Galβ1-4Glc1-F,
Neu5Acα2-8Neu5Acα2-3Galβ1-4Glc1-F,
Neu5Acα2-3Galβ1-3GalNAcβ1-4(Neu5Acα2-8Neu5Acα2-3)Galβ1-4Glc1-F,
Neu5Acα2-8Neu5Acα2-8Neu5Acα2-3Galβ1-4Glc1-F,
Neu5Acα2-8Neu5Acα2-3Galβ1-3GalNAcβ1-4(Neu5Acα2-8Neu5Acα2-3)Galβ1-4Glc1-F,
Neu5Acα2-3Gal1-F,

respectively, wherein the glycosyl moiety may be linked via an alpha (α) or a beta (β) glycosidic bond, preferably an alpha (α) glycosidic bond.

In some embodiments, the glycosyl moiety of the glycosyl fluoride is that of a human milk oligosaccharide, and wherein the human milk oligosaccharide is preferably selected from LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT.

In the context of the present invention glycosyl fluorides wherein the glycosyl moiety is that of LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT may be represented by the following formulas:

Galβ1-3GlcNAcβ1-3Galβ1-4Glc1-F,
Galβ1-4GlcNAcβ1-3Galβ1-4Glc1-F,
Galβ1-3GlcNAcβ1-3(Galβ1-4GlcNAcβ1-6)Galβ1-4Glc1-F,
Galβ1-4GlcNAcβ1-3Galβ1-4GlucNAcβ1-3Galβ1-4Glc1-F,
Fucα1-2Galβ1-4Glc1-F,
Galβ1-4(Fucα1-3)Glc1-F,
Fucα1-2Galβ1-4(Fucα1-3)Glc1-F,
Fucα1-2Galβ1-3GlcNAcβ1-3Galβ1-4Glc1-F,
Galβ1-3(Fucα1-4)GlcNAcβ1-3Galβ1-4Glc1-F,
Galβ1-3(Fucα1-3)GlcNAcβ1-3Galβ1-4Glc1-F,
Galβ1-3GlcNAcβ1-3Galβ1-4(Fucα1-3)Glc1-F,
Fucα1-2Galβ1-3(Fucα1-4)GlcNacβ1-3Galβ1-4Glc1-F,
Neu5Acα2-3Galβ1-4Glc1-F,
Neu5Acα2-6Galβ1-4Glc1-F,
Neu5Acα2-3Galβ1-4(Fucα1,3)Glc1-F,
Neu5Acα2-3Galβ1-3GlcNAcβ1-3Galβ1-4Glc1-F,
Galβ1-3(Neu5Acα2-6)GlcNAcβ1-3Galβ1-4Glc1-F,
Neu5Acα2-6Galβ1-3GlcNAcβ1-3Galβ1-4Glc1-F,
Neu5Acα2-3Galβ1-3(Neu5Acα2-6)GlcNAcβ1-3Galβ1-4Glc1-F,

respectively, wherein the glycosyl moiety may be linked via an alpha (α) or a beta (β) glycosidic bond, preferably an alpha (α) glycosidic bond.

As used herein, the term “protected glycosyl fluoride” refers to 1-deoxy-1-fluoro glycosides wherein a fluoride is covalently attached to the anomeric carbon at the reducing end of a protected glycosyl moiety. The fluoride may be bound to the anomeric carbon of the protected glycosyl moiety by either an alpha (α) or a beta (β) glycosidic linkage. An alpha glycosidic linkage is preferred.

The protected glycosyl moiety of the protected glycosyl fluoride may derive from a monosaccharide or from an oligosaccharide (more than one monosaccharide units). A glycosyl moiety having more than one monosaccharide unit may represent a linear or a branched structure.

The monosaccharide unit can be any C₅₋₉ sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

In some embodiments, all the hydroxyl groups of the glycosyl moiety of the protected glycosyl fluoride are derivatized with an acyl protecting group, wherein the acyl protecting group is selected from the group consisting of a C₂₋₆ acyl, or a benzoyl protecting group.

In some embodiments, all hydroxyl groups of the glycosyl moiety of the protected glycosyl fluoride are derivatized with an acyl protecting group, wherein the acyl protecting group is a C₂₋₆ acyl.

Accordingly in some embodiments the protected glycosyl fluoride is a per-O-acylated glycosyl fluoride.

In some embodiments, all hydroxyl groups of the glycosyl moiety of the protected glycosyl fluoride are derivatized with an acetyl protecting group. Accordingly in some preferred embodiments the protected glycosyl fluoride is a per-O-acetylated glycosyl fluoride.

The protected glycosyl fluoride may be an alpha (α) or a beta (β) glycoside, preferably an alpha (α) glycoside.

In some embodiments the protected glycosyl fluoride is selected from the group consisting of, glucosyl fluoride tetraacetate, galactosyl fluoride tetraacetate, and lactosyl fluoride heptaacetate, wherein the protected glycosyl fluoride may be an alpha (α) or a beta (β) glycoside, preferably is an alpha (α) glycoside.

In some preferred embodiments, the protected glycosyl fluoride is α-D-lactopyranosyl fluoride heptaacetate.

In some embodiments, the protected glycosyl fluoride is α-D-glucopyranosyl fluoride tetraacetate.

In some embodiments, the protected glycosyl fluoride is α-D-galactopyranosyl fluoride tetraacetate.

In some embodiments, the glycosyl moiety of the protected glycosyl fluoride is a protected derivative of the glycosyl moiety of a ganglioside preferably selected from GM1a, GM1b, GD1a, GD1b, GD3, GT1b, GT3, GQ1b, GM3, GM4, wherein all hydroxyl groups of the glycosyl moiety of the ganglioside are derivatized with an acyl protecting group, and wherein for those glycosyl moieties of gangliosides carrying a sialic acid unit, such as a N-acetylneuraminic acid unit, the carboxylic acid function of the sialic acid unit is in the form of an alkyl ester.

In some embodiments, all hydroxyl groups of the glycosyl moiety of the ganglioside are derivatized with an acetyl protecting group.

In some embodiments, the glycosyl moiety of the protected glycosyl fluoride is that of a human milk oligosaccharide preferably selected from LNT, LNnT, LNH, LNnH, 2′FL, 3FL, DFL, LNFP-1, LNFP-II, LNFP-III, LNFP-V, LNDFH-I, 3′SL, 6′SL, FSL, LSTa, LSTb, LSTc, and DSLNT, wherein all hydroxyl groups of the human milk oligosaccharide are derivatized with an acyl protecting group, and wherein for those oligosaccharides carrying a sialic acid unit, such as N-acetylneuraminic acid unit, the carboxylic function of the sialic acid unit is in the form of an alkyl ester.

In some embodiments all hydroxyl groups of the human milk oligosaccharide are derivatized with an acetyl protecting group.

The present invention describes a method for the production of a glycosyl fluoride, wherein the method comprising the steps of:
reacting the protected saccharide with a fluorinating agent, wherein the fluorinating agent is selected from the group consisting of pyridinium poly(hydrogen fluoride), triethylamine trihydrofluoride, and DMPU-HF, thereby obtaining a protected glycosyl fluoride,
deprotecting the protected glycosyl fluoride, thereby producing the glycosyl fluoride,
   and wherein the step of reacting the protected saccharide with the fluorinating agent is performed in the presence of a Lewis acid.

Lewis acids suitable for use in the context of the present invention include but are not limited to iodine, boron trifluoride, boron trichloride, aluminum chloride, aluminum bromide, zinc chloride, zinc bromide, zinc fluoride, zinc triflate, ferric chloride, ferric bromide, or complexes thereof. Further examples of Lewis acids include but are not limited to BF₃—HF, BF₃—NaCl, BCl₃—NaCl, BF₃—AgF, BF₃-nitrobenzene and the like.

In some embodiments, the Lewis acid is boron trifluoride, or a complex thereof.

In some embodiments, the Lewis acid is boron trifluoride diethyl etherate.

Typically, from about 1 to about 3 molar equivalents of the Lewis acid are used, based on the amount of the protected saccharide. Accordingly, in some embodiments the protected saccharide and the fluorinating agent are reacted in the presence of about 1.0, 1.1, 1.2, 1.3, 1.4, 1.6, 1.5, 1.7, 1.8, 1.9, 2.0, 2.1, 2.2, 2.3, 2.4, 2.5, 2.6, 2.7, 2.8, 2.9, or 3.0 equivalents of the Lewis acid, based on the amount of the protected saccharide.

Suitable fluorinating agents include but are not limited to pyridinium poly(hydrogen fluoride), tetrabutylammonium hydrogen difluoride, triethylamine trihydrofluoride, DMPU-HF, and the like. In some embodiment the fluorinating agent is pyridinium poly(hydrogen fluoride).

Typically, the fluorinating agent is used in an amount from about 1 to about 10 molar equivalents, based on the amount of the protected saccharide. In some embodiments, the fluorinating agent is used in an amount from about 3 to about 8 molar equivalents, based on the amount of the protected saccharide. Accordingly, in a some embodiments the fluorinating agent is used in an amount of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 molar equivalents, based on the amount of the protected saccharide.

The step of reacting the protected saccharide with the fluorinating agent is typically performed in a solvent such as dichloromethane, toluene, trifluorotoluene, tetrahydrofuran, 2-methyl-tetrahydrofuran, acetonitrile, or mixtures thereof.

In some embodiments, the step of reacting the protected saccharide with the fluorinating agent is performed in dichloromethane.

In some embodiments, the step of reacting the protected saccharide with the fluorinating agent is performed in toluene.

In some embodiments, the step of reacting the protected saccharide with the fluorinating agent is performed in a mixture of dichloromethane and toluene.

In some embodiments, the step of reacting the protected saccharide with the fluorinating agent is performed in acetonitrile.

The protected saccharide, the fluorinating agent, and the Lewis acid are typically reacted at a temperature from about −5° C. to about 5° C., over about 2 to about 4 hours. Preferably, from about 3 to about 4 hours.

The reaction between the protected saccharide and the fluorinating agent in the presence of the Lewis acid, results in the replacement of the carbon-oxygen bond at the anomeric position of the protected saccharide by a carbon-fluorine bond. The reaction between the protected saccharide and the fluorinating agent may also be referred to as “fluorination”.

The fluorination of a protected saccharide with a fluorinating agent in the presence of the Lewis acid results in the formation of a protected glycosyl fluoride.

In some embodiments, the protected glycosyl fluoride is a per-O-acylated glycosyl fluoride. Accordingly, in some embodiments, the step of deprotecting the protected glycosyl fluoride is a deacylation step.

In some embodiments, the protected glycosyl fluoride is a per-O-acetylated glycosyl fluoride. Accordingly, in some embodiments, the step of deprotecting the protected glycosyl fluoride is a deacetylation step.

The step of deprotecting the protected glycosyl fluoride is typically performed in the presence of a base. Examples of suitable bases include but are not limited to alkali metal alkoxides such as those deriving from methanol, ethanol, propanol, isopropanol, butanol, and isobutanol, wherein the alkali metal is selected from sodium, potassium, or lithium.

In some embodiments the step of deprotecting the protected glycosyl fluoride is performed in the presence of sodium methoxide, or sodium ethoxide.

In some preferred embodiments, the step of deprotecting the protected glycosyl fluoride is performed in the presence of sodium methoxide.

The base may be used in catalytic amounts, equimolar amounts or in excess. Preferably, the base is used in an amount from about 0.1 to about 2 molar equivalents.

In some embodiments, the base is used in an amount from about 0.2 to about 0.5 molar equivalents. Accordingly in some embodiments, the base is used in the amount of about 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.5 molar equivalents.

The step of deprotecting the protected glycosyl fluoride is typically performed in a solvent. Examples of suitable solvents include but are not limited to methanol, ethanol, propanol, isopropanol, butanol, isobutanol and the like.

In some embodiments, the step of deprotecting the protected glycosyl fluoride is performed in methanol.

In some embodiments, the step of deprotecting the protected glycosyl fluoride is performed in ethanol.

The components of the reactions of the invention may be combined in any order, and it will be appreciated that the order of combining the reactants may be adjusted as needed.

The glycosyl fluoride produced by the above method can be used without purification. However, in some embodiments, the glycosyl fluoride may be purified.

The purification of the glycosyl fluoride may be performed by standard methods known to the skilled person, such as for example extraction with organic solvents, chromatography, crystallization, or precipitation.

A preferred method of purification involves the precipitation of the glycosyl fluoride. The precipitation of the glycosyl fluoride may be achieved for example via partial removal of the reaction solvent by evaporation i.e., concentrating the rection mixture, or via the addition of another solvent to the reaction mixture, or via changes of temperature or pressure, or via addition of other solutes, or combinations of these.

The glycosyl fluoride according to the present invention, may be produced in different polymorphic forms. Polymorphic forms, as referred to herein, can include crystalline and amorphous forms as well as solvate and hydrate forms, which can be further characterized as follows:
Crystalline forms have different arrangements and/or conformations of the molecules in the crystal lattice.
Amorphous forms consist of disordered arrangements of molecules that do not possess a distinguishable crystal lattice.
Solvates are crystal forms containing either stoichiometric or non-stoichiometric amounts of a solvent. If the incorporated solvent is water, the solvate is commonly known as a hydrate.

In some embodiments, the glycosyl fluoride is obtained in the form of a solvate. In some embodiments, the glycosyl fluoride is obtained in the form of a hydrate, such as in the form of monohydrates, dihydrates or trihydrates. In some embodiments, the glycosyl fluoride is obtained in a crystalline form. In some embodiments, the glycosyl fluoride is obtained in an amorphous form.

In some embodiments, the protected saccharide is per-O-acylated saccharide and the method further comprising a step of producing the per-O-acylated saccharide. Accordingly in some embodiments, the method comprising the steps of:
reacting a saccharide with a compound of formula (1):

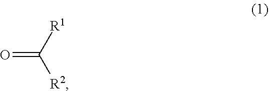

wherein
R¹ is a phenyl, preferably an unsubstituted phenyl, or a C₁₋₆ alkyl,
R² is —CI, or —OC(═O)R³, wherein R³ is a phenyl, preferably an unsubstituted phenyl, or a C₁₋₆ alkyl,
in the presence of an aliphatic amine in an aprotic solvent, thereby obtaining per-O-acylated saccharide;
reacting the per-O-acylated saccharide with a fluorinating agent, wherein the fluorinating agent is selected from the group consisting of pyridinium poly(hydrogen fluoride), triethylamine trihydrofluoride, and DMPU-HF, thereby obtaining a per-O-acylated glycosyl fluoride,
deprotecting the per-O-acylated glycosyl fluoride, thereby producing the glycosyl fluoride, and wherein the step of reacting the per-O-acylated saccharide with the fluorinating agent is performed in the presence of a Lewis acid.

In some embodiments, the compound of formula (1) is acetic anhydride. Accordingly in some embodiments, the step of reacting a saccharide with a compound of formula (1) is an O-acetylation step.

In some embodiments, the compound of formula (1) is benzoyl chloride. Accordingly in some embodiments, the step of reacting a saccharide with a compound of formula (1) is an O-benzoylation step.

The reaction between the saccharide and the compound of formula (1) is typically performed in the presence of an aliphatic amine.

In some embodiments, the aliphatic amine is selected from triethyl amine or N,N-diisopropylethylamine,

In some embodiments, the aliphatic amine is triethyl amine.

The reaction between the saccharide with the compound of formula (1) is typically performed in an aprotic solvent.

In some embodiments, the solvent is selected from acetone, dichloromethane, toluene, and acetonitrile.

In some embodiments, the solvent is acetone.

In some embodiments, the solvent is dichloromethane.

In some embodiments, the solvent is acetonitrile.

The temperature at which the above process is carried out may range from room temperature to the temperature corresponding to the boiling point of the selected solvent. That temperature range is preferably at about 25° C. to about 120° C.

In some embodiments the O-acylation step is carried out using acetone as the solvent, and the reaction is carried out at a temperature between about 25° C. to about 60° C. Preferably between about 50° C. to about 60° C. In some embodiments, the O-acylation step is carried out at a temperature of about 50° C., 51° C., 52° C., 53° C., 54° C., 55° C., 56° C., 57° C., 58° C., 59° C., or 60° C.

In some embodiments, the O-acylation step is carried out using dichloromethane as the solvent, and the reaction is carried out at a temperature between about 25° C. to about 45° C. Preferably between about 35° C. to about 45° C. In some embodiments, the O-acylation is carried out at a temperature of about 35° C., 36° C., 37° C., 38° C., 39° C., 40° C., 41° C., 42° C., 43° C., 44° C. or 45° C.

In some embodiment, the O-acylation step is carried out using toluene as the solvent, and the reaction is carried out at a temperature between about 100° C. to about 120° C. Preferably between about 100° C. to about 110° C. In some embodiments, the O-acylation step is carried out at a temperature of about 100° C., 101° C., 102° C., 103° C., 104° C., 105° C., 106° C., 107° C., 108° C., 109° C. or 110° C.

The O-acylation step is allowed to proceed for a period of time sufficient to obtain the desired high yield of the desired O-acylated product. Typically, the reaction is allowed to proceed for between about 1 to about 24 hours, preferably between about 4 to about 24 hours. In some embodiments, reaction is allowed to proceed for about 4, 5, 6, 7, 8, 9, 10, 11, 12, 13, 14, 15, 16, 17, 18, 19, 20, 21, 22, 23 or 24 hours.

The components of the reactions of the invention may be combined in any order, and it will be appreciated that the order of combining the reactants may be adjusted as needed.

The per-O-acylated saccharide produced by the above method can be used without purification. However, in some embodiments, the per-O-acylated saccharide may be purified.

The step of purifying the per-O-acylated saccharide may be performed by standard methods known to the skilled person, such as for example extraction with organic solvents, chromatography, crystallization, or precipitation.

A preferred method of purification involves the precipitation of the per-O-acylated saccharide. The precipitation of the per-O-acylated saccharide may be achieved directly from the reaction mixture, by cooling the reaction mixture or by the adding an antisolvent to the reaction mixture. Alternatively, the precipitation may be performed after extraction with an organic solvent followed by cooling or adding an antisolvent to the solution containing the per-O-acylated saccharide glycosyl ester, or a combination of these.

The per-O-acylated saccharide according to the present invention, may be produced in different polymorphic forms. Polymorphic forms, as referred to herein, can include crystalline and amorphous forms as well as solvate and hydrate forms, which can be further characterized as follows:
Crystalline forms have different arrangements and/or conformations of the molecules in the crystal lattice.
Amorphous forms consist of disordered arrangements of molecules that do not possess a distinguishable crystal lattice.
Solvates are crystal forms containing either stoichiometric or non-stoichiometric amounts of a solvent. If the incorporated solvent is water, the solvate is commonly known as a hydrate.

In some embodiments, the per-O-acylated saccharide is obtained in the form of a solvate. In some embodiments, the per-O-acylated saccharide is obtained in the form of a hydrate, such as in the form of monohydrates, dihydrates or trihydrates. In some embodiments, the per-O-acylated saccharide is obtained in a crystalline form. In some embodiments, the per-O-acylated saccharide is obtained in an amorphous form.

### EXAMPLES

Working examples below describe non-limiting embodiments of the invention and are given only to illustrate the invention.

### General Methods and Material:

¹H NMR was recorded with a JEOL (500.16 MHz) spectrometer, or a Bruker Avance II (400 MHz) spectrometer. ¹H chemical shifts are given in ppm (δ) relative to tetramethylsilane (δ=0.00), D₂O (δ=4.79), or CDCl3 (δ=7.26, δ=77.00) as internal standard. MS analysis was performed with a Shimadzu LCMS-2020 system.

Thin layer chromatography (TLC) was performed with silica gel TLC-plates (Merck, Silica gel, F254) with detection by UV-absorption (254 nm) where applicable and carrying (140° C.) with ammonium molybdate (25 g/L) and cerium ammonium sulfate (10 g/L) in 10% H₂SO₄.

### Example 1. General Method for the O-Acylation of Saccharides

The saccharide (1 eq.) and triethylamine (4-16 eq.) were suspended in acetone, dichloromethane, or toluene. The suspension was heated at reflux and acetic anhydride (6-18 eq.), or benzoyl chloride (6-10 eq.), was added to the reaction mixture. The mixture was heated at reflux until TLC showed complete consumption of the starting material. The reaction mixture was cooled to room temperature until the product precipitated. If necessary, water was added to the cooled reaction mixture to facilitate the precipitation. The resulting solid was filtered, washed with water, and dried in vacuum. The O-acylated product may be crystallized from MeOH. The O-acylated product is typically obtained in about 65-85% yield.

### Example 2. Production of β-D-Lactose Octaacetate

β-D-Lactose octaacetate was produced from lactose following the general method described in Example 1 using acetone as the solvent.

Anomeric ratio 1:10 alpha:beta

¹H NMR (400 MHz, CDCl₃): δ (ppm)=1.94 (s, 3H), 2.00 (s, 3H), 2.02 (s, 3H), 2.03 (s, 3H), 2.04 (s, 3H), 2.07 (s, 3H), 2.09 (s, 3H), 2.13 (s, 3H), 3.69-3.78 (m, 2H), 3.78-3.90 (m, 2H), 4.01-4.16 (m, 3H), 4.39-4.49 (m, 2H), 4.92 (dd, J=10.1, 3.4 Hz, 1H), 5.08 (t, J=9.2 Hz, 1H), 5.22 (t, J=9.2 Hz, 1H), 5.32 (d, J=2.6 Hz, 1H), 5.65 (d, J=8.2 Hz, 1H).

### Example 3. Production of β-D-Lactose Octabenzoate

β-D-Lactose octabenzoate was produced from lactose following the general method described in Example 1 using acetone as the solvent.

Anomeric ratio 1:4 alpha:beta

¹H NMR (400 MHz, CDCl₃): δ (ppm)=3.71 (dd, J=11.3, 7.1 Hz, 1H) 3.78 (dd, J=11.3, 6.4 Hz, 1H), 3.89 (t, J=7.0 Hz, 1H), 4.03-4.11 (m, 1H), 4.39 (t, J=9.4 Hz, 1H), 4.53 (dd, J=12.4, 3.8 Hz, 1H), 4.59 (dd, J=12.4, 1.8 Hz, 1H), 4.89 (d, J=7.9 Hz, 1H), 5.38 (dd, J=10.4, 3.3 Hz, 1H), 5.71-5.716 (m, 2H), 5.78 (dd, J=9.6, 8.1 Hz, 1H), 5.94 (t, J=9.3 Hz, 1H), 6.14 (d, J=8.1 Hz, 1H), 7.96-8.08 (m, 16H), 7.29-7.65 (m, 24H).

### Example 4. Production of β-D-Galactopyranosyl Pentaacetate

β-D-Galactose pentaacetate was produced from galactose following the general method described in Example 1 using dichloromethane as the solvent.

Anomeric ratio 1:6.5 alpha:beta

¹H NMR (400 MHz, MeOD): δ (ppm)=1.99 (s, 3H), 2.04 (s, 6H). 2-12 (s, 3H), 2.16 (s, 3H), 4.05 (ddd, J=7.1, 6.1, 1.2 Hz, 1H), 4.07-4.20 (m, 2H), 5.07 (dd, J=10.4, 3.4 Hz, 1H), 5.33 (dd, J=10.4, 8.3 Hz, 1H), 5-42 (dd, J=3.5, 1.2 Hz, 1H), 5.69 (d, J=8.3 Hz, 1H).

### Example 5. Synthesis of β-D-Lacto-N-neotetraosyl tetradecaacetate

β-D-lacto-N-neotetraosyl tetradecaacetate was produced from lacto-N-neotetraose following the general method described in Example 1 using toluene as the solvent.

Anomeric ratio 1:9 alpha:beta

¹H NMR (400 MHz, CDCl₃): δ (ppm)=1.88 (s, 3H), 1.95 (s, 3H), 2.01 (s, 6H), 2.02 (s, 3H), 2.04 (s, 3H), 2.05 (s, 3H), 2.05 (s, 3H), 2.08 (s, 3H), 2.09-2.10 (m, 6H), 2.11 (s, 3H), 2.13 (s, 3H), 2.13 (s, 3H), 3.47-3.52 (m, 2H), 3.71 (dd, J=10.0, 3.6 Hz, 1H), 3.73-3.82 (m, 4H), 3.86 (ddd, J=7.5, 6.2, 1.3 Hz, 1H), 3.95 (dd, J=12.0, 3.3 Hz, 1H), 3.99-4.16 (m, 5H), 4.32 (d, J=8.0 Hz, 1H), 4.40 (dd, J=12.1, 1.9 Hz, 1H), 4.53 (d, J=7.9 Hz, 1H), 4.66 (d, J=7.9 Hz, 1H), 4.76 (dd, J=12.0, 2.6 Hz, 1H), 4.94-4.99 (m, 2H), 5.02 (dd, J=9.5, 8.3 Hz, 1H), 5.09 (dd, J=10.5, 7.9 Hz, 1H), 5.14-5.22 (m, 2H), 5.29 (dd, J=3.6, 1.1 Hz, 1H), 5.33 (dd, J=3.4, 1.2 Hz, 1H), 5.38 (d, J=8.6 Hz, 1H), 5.65 (d, J=8.3 Hz, 1H).

LCMS: 1254 [M+H]⁺, 1276 [M+Na]⁺.

### Example 6. Synthesis of β-D-Lacto-N-tetraosyl tetradecaacetate

β-D-lacto-N-tetraosyl tetradecaacetate was synthesised from lacto-N-tetraose following the general method described in Example 1 using toluene as the solvent.

Anomeric ratio 1:8 alpha:beta

¹H NMR (400 MHz, CDCl₃): δ (ppm)=1.95 (s, 3H), 1.98 (s, 3H), 2.01 (s, 6H), 2.03 (s, 3H), 2.04 (s, 3H), 2.06 (s, 3H), 2.08 (s, 3H), 2.08 (s, 6H), 2.09 (s, 3H), 2.10 (s, 3H), 2.11 (s, 3H), 2.13 (s, 3H), 3.60 (ddd, J=10.1, 4.0, 2.7 Hz, 1H), 3.72-3.80 (m, 4H), 3.83 (td, J=6.7, 1.2 Hz, 1H), 3.98 (dd, J=11.6, 3.6 Hz, 1H), 4.01-4.09 (m, 4H), 4.15 (dd, J=12.0, 4.3 Hz, 1H), 4.33 (d, J=7.8 Hz, 1H), 4.36 (dd, J=12.1, 2.6 Hz, 1H), 4.39-4.45 (m, 2H), 4.57 (dd, J=10.5, 9.2 Hz, 1H), 4.87-4.97 (m, 3H), 4.99-5.05 (m, 3H), 5.11 (d, J=8.1 Hz, 1H), 5.22 (dd, J=9.5, 8.8 Hz, 1H), 5.30-5.34 (m, 2H), 5.66 (d, J=8.3 Hz, 1H), 5.88 (d, J=6.9 Hz, 1H).

LCMS: 1254 [M+H]⁺, 1276 [M+Na]⁺.

### Example 7. Production of β-D-Melibiose octaacetate

β-D-Melibiose octaacetate was produced form melibiose following the general method described in Example 1, using acetone as the solvent.

¹H NMR (400 MHz, CDCl₃): δ (ppm)=1.98 (s, 3H), 2.00 (s, 3H), 2.02 (s, 3H), 2.03 (s, 3H), 2.04 (s, 3H), 2.09 (s, 3H), 2.12 (s, 3H), 2.13 (s, 3H), 3.62 (dd, J=11.6, 2.5 Hz, 1H), 3.71 (dd, J=11.6, 4.4 Hz, 1H), 3.77 (ddd, J=9.8, 4.4, 2.5 Hz, 1H), 4.01-4.10 (m, 2H), 4.17 (td, J=6.4, 1.3 Hz, 1H), 5.01-5.18 (m, 4H), 5.24 (t, J=9.4 Hz, 1H), 5.33 (dd, J=10.8, 3.4 Hz, 1H), 5.44 (dd, J=3.4, 1.3 Hz, 1H), 5.66 (d, J=8.3 Hz, 1H).

LCMS: 696 [M+NH₄]⁺, 701 [M+Na]⁺.

### Example 8. General Procedure for the Production of Glycosyl Fluorides

A per-O-acetylated saccharide (1 eq.) was dissolved dichloromethane (5 vol.). The solution was cooled down to a temperature between about −10 to about −5° C.

Pyridinium poly(hydrogen fluoride) (70%, 8 eq.) and BF₃·Et₂O (2.2 eq) were added, and the reaction mixture was warmed up to a temperature between about 0° C. to about 10° C. Upon completion, the reaction mixture was poured into ice cold water, the organic phase was separated, washed with water, saturated with NaHCO₃, and filtered through Na₂SO₄.

The solvent was evaporated, and methanol was added. The reaction mixture was then cooled to room temperature and NaOMe (25 wt % in methanol, 0.05 eq.) was added. The mixture was stirred until formation of a white solid. The resulting suspension was stirred at a temperature between about 0° C. to about 25° C. over 1 hour. The obtained solid was filtered, washed with methanol, ethanol, or acetonitrile, and dried to obtain the glycosyl fluoride. Typical yield ranges from 60-85%.

### Example 9. Production of α-D-Lactopyranosyl fluoride

α-D-Lactopyranosyl fluoride was produced from β-D-lactose octaacetate following the procedure described in Example 8.

¹H-NMR (500 MHz, D₂O): 5.59 (dd, J=53.5 Hz, 2.8 Hz, 1H) 4.36 (d, J=7.7 Hz, 1H), 3.88-3.74 (m, 5H), 3.73-3.52 (m, 6H), 3.45 (dd, J=10.0, 7.7 Hz, 1H).

LCMS: 367 [M+Na]⁺, 408 [M+NH₄HCO₂+H]⁺.

### Example 10. Production of α-D-Galactopyranosyl fluoride

α-D-Galactopyranosyl fluoride was produced from β-D-galactopyranosyl pentaacetate following the procedure described in Example 8.

¹H NMR (400 MHz, CD₃OD): δ=5.58 (d, J=54.4 Hz, 1H), 3.99-3.94 (m, 2H), 3.87-3.71 (m, 4H).

### Example 11. Production of α-D-Glucopyranosyl fluoride

α-D-Glucopyranosyl fluoride was produced from β-D-glucopyranosyl pentaacetate following the procedure described in Example 8.

¹H NMR (400 MHz, CD₃OD): δ=5.55 (dd, J=53.9, 2.8 Hz, 1H), 3.83 (m, 1H), 3.49-3.38 (m, 2H).

### Example 12. Production of α-D-melibiosyl fluoride

α-D-Melibiosyl fluoride was produced from β-D-Melibiose octaacetate following the procedure described in Example 8.

¹H-NMR (400 MHz, MeOD): δ=3.39-3.57 (m, 3H), 3.61-3.83 (m, 8H), 3.85-4.02 (m, 5H), 5.56 (dd, J=53.7, 2.7 Hz, 1H).

LCMS: 408 [M+CH₃CN+Na]⁺.

### Example 13. Production of α-D-Lacto-N-neotetraosyl fluoride

α-D-Lacto-N-neotetraosyl fluoride was produced from β-D-Lacto-N-neotetraosyl tetradecaacetate following the procedure described in Example 8.

¹H NMR (500 MHz, D₂O): δ=2.05 (s, 3H), 3.51-3.92 (m, 21H), 3.92-4.02 (m, 4H), 4.17 (d, J=3.3 Hz, 1H), 4.48 (dd, J=13.2, 7.8 Hz, 2H), 4.73 (d, J=8.3 Hz, 1H), 5.71 (dd, J=53.5, 2.7 Hz, 1H).

LCMS: 732 [M+Na]⁺.

### Example 14. Production of α-D-lacto-N-tetraosyl fluoride

α-D-Lacto-N-tetraosyl fluoride was produced from β-D-Lacto-N-tetraosyl tetradecaacetate following the procedure described in Example 8.

¹H-NMR (400 MHz, D₂O): δ=2.03 (s, 3H), 3.45-3.68 (m, 6H), 3.68-4.00 (m, 17H), 4.16 (d, J=3.3 Hz, 1H), 4.46 (dd, J=7.8, 5.7 Hz, 2H), 4.74 (d, J=8.3 Hz, 1H), 5.70 (dd, J=53.6, 2.7 Hz 1H).

LCMS: 710 [M+H]⁺, 732 [M+Na]⁺.

The above-described embodiments are combinable. The following dependent claims set out particular embodiments of the invention.

1. Method for producing a glycosyl fluoride by the fluorination of a protected saccharide, wherein each hydroxyl group of said protected saccharide is derivatized with a protecting group, and wherein the anomeric hydroxyl group of said protected saccharide is derivatized with an acyl protecting group,
the method comprising the steps of:
reacting the protected saccharide with a fluorinating agent, wherein the fluorinating agent is selected from the group consisting of pyridinium poly(hydrogen fluoride), triethylamine trihydrofluoride, and DMPU-HF, thereby obtaining a protected glycosyl fluoride,
deprotecting the protected glycosyl fluoride, thereby producing the glycosyl fluoride,
and wherein the step of reacting the protected saccharide with the fluorinating agent is performed in the presence of a Lewis acid.

2. The method according to claim 1, wherein the fluorinating agent is pyridinium poly(hydrogen fluoride).

3. The method according to claim 2, wherein about 1 to about 10 molar equivalents of the pyridinium poly(hydrogen fluoride) are used, based on the amount of the protected saccharide.

4. (canceled)

5. The method according to claim 1, wherein the Lewis acid is boron trifluoride or a complex thereof.

6. (canceled)

7. (canceled)

8. The method according to claim 1, wherein the step of reacting the protected saccharide with the fluorinating agent further comprises the use of a solvent.

9. The method according to claim 8 wherein the solvent is selected from the group consisting of dichloromethane, toluene, trifluorotoluene, tetrahydrofuran, 2-metyltetrahydrofuran acetonitrile, or mixtures thereof.

10. The method according to claim 1 wherein step of deprotecting the protected glycosyl fluoride is performed in the presence of a base.

11. The method according to claim 10, wherein the base is an alkoxide, and wherein the alkoxide is selected from sodium methoxide, or sodium ethoxide.

12. (canceled)

13. (canceled)

14. The method according to claim 1, wherein the protected saccharide is a beta (β) saccharide, and wherein the glycosyl fluoride is an alpha (α) glycosyl fluoride.

15. The method according to claim 1, wherein the glycosyl fluoride is selected from the group consisting of glucopyranosyl fluoride, galactopyranosyl fluoride, and lactopyranosyl fluoride.

16. (canceled)

17. The method according to claim 1, wherein the protected saccharide is a per-O-acylated saccharide.

18. (canceled)

19. The method according to claim 17, wherein the per-O-acylated saccharide is a per-O-acetylated saccharide.

20. The method according to claim 17, wherein the per-O-acylated saccharide is selected from the group consisting of glucose pentaacetate, galactose pentaacetate, and lactose octaacetate.

21. The method according to claim 17, wherein the method further comprising a step of producing a per-O-acylated saccharide.

22.-29. (canceled)
